(19) United States
(12) Patent Application Publication
(10) Pub. No.: US 2024/0156419 A1
(43) Pub. Date: May 16, 2024

(54) SYSTEM FOR GAIT TRAINING AND METHOD THEREOF

(71) Applicant: Nanyang Technological University, Singapore (SG)

(72) Inventors: Xi FU, Singapore (SG); Neethu ROBINSON, Singapore (SG); Cuntai GUAN, Singapore (SG)

(73) Assignee: Nanyang Technological University, Singapore (SG)

(21) Appl. No.: 18/506,266

(22) Filed: Nov. 10, 2023

(30) Foreign Application Priority Data
Nov. 14, 2022 (SG) 10202260066V

(51) Int. Cl. A61B 5/00; A61B 5/11; A61B 5/291

(52) U.S. Cl. CPC A61B 5/7275; A61B 5/112; A61B 5/291; A61B 5/7267

(57) ABSTRACT

Disclosed herein is a system for gait training and a method of gait training. The system includes a gait prediction module configured to: using a two-stage machine learning model, extract gait features from EEG signals acquired from a subject; and determine a predicted gait based on the gait features, wherein the two-stage machine learning model includes multiple first stage blocks and multiple second stage blocks, the first stage blocks and the second stage blocks being trained based on different gait data obtained solely from the subject at different time points, wherein at least one of the second stage blocks is a feature extractor block, each of the feature extractor block corresponding to respective ones of the first stage blocks.

### CROSS-REFERENCE TO RELATED APPLICATION

This application claims the benefit of priority to the Singapore application no. 10202260066V filed Nov. 14, 2022, the contents of which are hereby incorporated by reference in their entirety for all purposes.

### TECHNICAL FIELD

This application relates to a system for gait training and a method of gait training.

### BACKGROUND

Stroke is one of the primary causes of persistent physical or cognitive disabilities, with an estimated fifty percent of survivors suffering from such impairments. Hemiplegia or hemiparesis is the most prevalent and debilitating symptom after a stroke, and it has a substantial impact on lower limb function. In addition to rehabilitation interventions, it is also desirable for rehabilitation interventions to take into account the heterogeneity of outcomes, thus allowing the prognosis of treatment-induced motor recovery in stroke care and planning.

### SUMMARY

According to an aspect, disclosed herein a system for gait training. The system includes: memory storing instructions; and a processor coupled to the memory and configured to process the stored instructions to implement: a gait prediction module configured to: using a two-stage machine learning model, extract gait features from EEG signals acquired from a subject; and determine a predicted gait based on the gait features. In some embodiments, the two-stage machine learning model may include multiple first stage blocks and multiple second stage blocks, the first stage blocks and the second stage blocks being trained based on different gait data obtained solely from the subject at different time points. In some embodiments, at least one of the second stage blocks is a feature extractor block, each of the feature extractor block corresponding to respective ones of the first stage blocks.

According to another aspect, a method of gait training is disclosed. The method includes: using a two-stage machine learning model, extracting gait features from EEG signals acquired from a subject; and determining a predicted gait based on the gait features. In some embodiments, the two-stage machine learning model includes multiple first stage blocks and multiple second stage blocks, the first stage blocks and the second stage blocks being trained based on different gait data obtained solely from the subject at different time points. In some embodiments, at least one of the second stage blocks is a feature extractor block, each of the feature extractor block corresponding to respective ones of the first stage blocks.

### BRIEF DESCRIPTION OF THE DRAWINGS

Various embodiments of the present disclosure are described below with reference to the following drawings:

FIG. 1 is a schematic diagram of a system for gait training according to embodiments of the present disclosure;

FIGS. 2A and 2B are examples of visual representations of a subject according to various embodiments;

FIG. 3 is a schematic diagram of a two-stage machine learning model according to various embodiments;

FIGS. 4A and 4B are schematic diagrams of a two-stage machine learning model according to various embodiments;

FIG. 5 is a flow chart of a method for gait training according to various embodiments;

FIG. 6 is a schematic diagram of a teacher model of a two-stage machine learning model according to an example;

FIG. 7 is a schematic diagram of a student model of the two-stage machine learning model of FIG. 6;

FIG. 8 is a schematic diagram illustrating a partition of dataset for trials 1, 2 and 3;

FIG. 9 shows the results of different models, with the X-axis as the tap size, the Y-axis as the average r-values;

FIG. 10 shows the results of comparison between MATN model and the two variant models (MMN and SAM) in terms of average r-values for various tap sizes; and

FIG. 11 is a block diagram representative of components of a processing system according to various embodiments of the invention.

### DETAILED DESCRIPTION

The following detailed description is made with reference to the accompanying drawings, showing details and embodiments of the present disclosure for the purposes of illustration. Features that are described in the context of an embodiment may correspondingly be applicable to the same or similar features in the other embodiments, even if not explicitly described in these other embodiments. Additions and/or combinations and/or alternatives as described for a feature in the context of an embodiment may correspondingly be applicable to the same or similar feature in the other embodiments.

In the context of various embodiments, the articles “a”, “an” and “the” as used with regard to a feature or element include a reference to one or more of the features or elements.

In the context of various embodiments, the term “about” or “approximately” as applied to a numeric value encompasses the exact value and a reasonable variance as generally understood in the relevant technical field, e.g., within 10% of the specified value.

As used herein, the term “and/or” includes any and all combinations of one or more of the associated listed items.

Further, one skilled in the art will recognize that many functional units in this description have been labelled as modules throughout the description. The person skilled in the art will also recognize that a module may be implemented as circuits, logic chips or any sort of discrete component, and multiple modules may be combined into a single module or divided into sub-modules as required without departing from the disclosure. Still further, one skilled in the art will also recognize that a module may be implemented in software which may then be executed by a variety of processors. In embodiments of the disclosure, a module may also comprise computer instructions or executable codes that may instruct a computer processor to carry out a sequence of events based on instructions received. The choice of the implementation of the modules is left as a decision to a person skilled in the art and does not limit the scope of this disclosure in any way.

For the sake of brevity, the term “machine learning model” may be used to refer to any one or more of the terms “artificial intelligence model”, “attention model”, “deep learning model”, “Multi-model Attention Network (MATN)”, etc., as will be understood from the context.

As used herein, the term “machine learning blocks” or “blocks” generally refers to the functional blocks which form part of or whole of a machine learning model. The machine learning blocks may include bias, parameters or weights which are variable or updateable during training of the respective machine learning blocks. The machine learning blocks may be connected in series to and/or in parallel with each other in a machine learning model. Therefore, one or more connected machine learning blocks may be trained collectively. As examples, the machine learning blocks may include Temporal Convolution Blocks (TCB), Spatial Convolution Blocks (SCB), Attention Blocks, separable convolution blocks, Fully connected (FC) layers, input/output blocks, etc. In some examples, the machine learning blocks may include one or more inputs and one or more outputs.

For the sake of brevity, the terms “EEG signal” or “EEG data” will generally refer to Electroencephalography (EEG) signals or EOG Electrooculograms (EOG) signals, or a combination thereof, obtained from one or more subjects corresponding to brain activity.

Challenges may be present in using EEG signals for gait training or gait rehabilitation. Firstly, due to the low signal-to-noise ratio of EEG signals, accurate regression requires the help of effective predictors or regression algorithms. Further, EEG signal varies from subject to subject due to individual differences, and hence the performance of predictors deteriorates when applied to different subjects. In addition, EEG signal is a dynamic motor performance of the subject, and such motor performance may vary from person to person. Therefore, the predictor may be required to process the time series dynamically. Lastly, variation in the EEG signal from the subject across multiple gait sessions may degrade the predictor performances.

Disclosed herein is a gait training system and a gait training method according to various embodiments. The gait training system and method may be used in gait training/rehabilitation and/or gait assessment for a subject. In an aspect, the system includes a gait prediction from electroencephalogram (EEG) signals/data and/or EOG Electrooculograms (EOG) signals/data acquired from a subject/user. The system may further include a real-time visual feedback-based gait training for the subject, and a gait training/rehabilitation metric for gait recovery/rehabilitation assessment. In various embodiments, a proposed deep learning model is employed to decode the brain (EEG and EOG signals) activity when the subject is walking and accurately predicts the subject's gait patterns. A comparison showed that the regression on the subject's EEG signals performed by the proposed model resulted in a Pearson's correlation coefficient of 0.752 with the actual gait of the subject. In another aspect, integration of the gait prediction with a virtual avatar serves as a visual feedback or stimulus for the subject during gait training/rehabilitation. The predicted gait pattern is translated to control an avatar, which simulates the subject's walking action. This closed-loop training strategy allows users to monitor how well their brain activity is translated to the avatar gait, and modulate the brain activity to minimize the error between predicted and expected (normal) gait patterns. In another aspect, a new metric for the assessment of gait training/rehabilitation and recovery status of the user is disclosed. The metric quantifies the state of the patient's gait recovery based on the deviation between gait predicted by the proposed technique from user's EEG and the normal gait. The modulations in this metric over time will be an indicator of efficacy of the proposed training/rehabilitation.

The gait training system/method may include a regression feedback method based on EEG signals to predict user's gait patterns and translate the EEG features to control an avatar which serves as visual feedback. Further, the gait training the system/method may include a rehabilitation assessment method based on feedback from real-time gait simulation, in which the predicted gait is obtained by continuously decoding the user's EEG signal, and the gait rehabilitation status is assessed based on the deviation between the predicted gait and the actual gait. Further, the gait training system/method may include a collaborative feedback method that trains subjects to perform accurate motor imagery based on visual feedback. This may be done by utilizing machine learning and deep learning methods for continuous regression of subjects' gait. Further, a visual neurofeedback-based assessment procedure is proposed by collecting, analyzing, and extracting gait-related information from brain signals.

To aid understanding and not to be limiting, various embodiments of a gait training system 100 and a gait training method 700 will be described below with reference to the appended figures. The gait training system 100 and gait training method 700 may also be described as a system and a method for assisting in gait training and gait assessment. For the sake of brevity, the following will describe various examples with respect to the system and method, but it will be understood that the system 100 and the method 700 may be used in multiple application scenarios pertaining to gait training and gait assessment and is not limited to the specific examples disclosed herein.

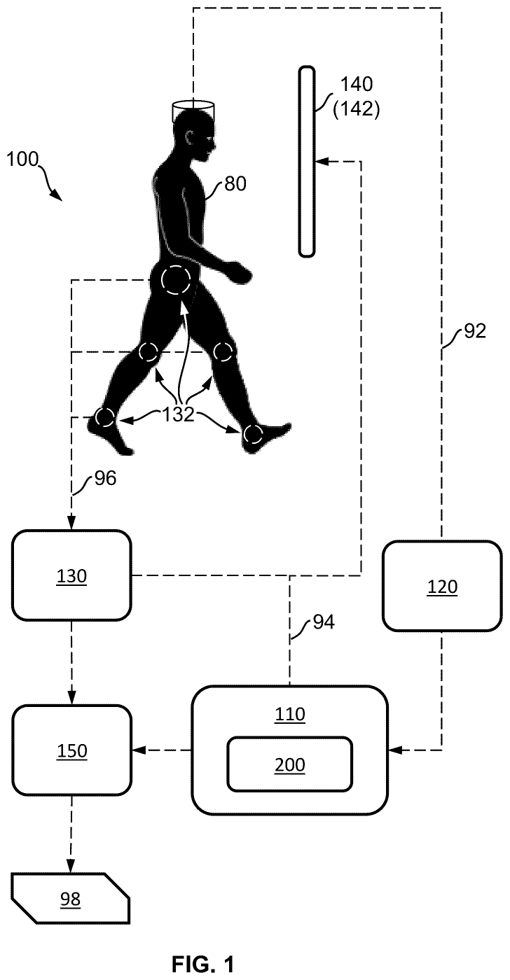

FIG. 1 is a schematic diagram of a system 100 for gait training. The system 100 may include a processing system which includes memory storing instructions; and a processor coupled to the memory and configured to process the stored instructions to implement various modules. In some embodiments, the processor is configured to implement a gait prediction module 110. The gait prediction module 110 may be used for gait prediction. The gait prediction module 110 may be in signal communication with an EEG acquisition module 120. The EEG acquisition module 120 may include multiple electrodes/channels, configured to acquire or collect EEG signals 92 and/or EOG signals from a subject 80.

In some embodiments, the gait prediction module 110 may be configured to extract gait features from EEG signals acquired from the subject 80 and determine a predicted gait based on the gait features. In various embodiments, the gait prediction module 110 may be a subject-specific module customized based on specific subjects. In some embodiments, the gait prediction module 110 may be iteratively trained, updated or improved upon each gait session, such as gait training session, gait assessment session or system calibration session. In some embodiments, the gait prediction module 110 may use a two-stage machine learning model 200 for extracting gait features from EEG signals acquired from a subject. The gait prediction module 110 may also use the two-stage machine learning model 200 for determining a predicted gait 94 based on the gait features. The predicted gait 94 may include a prediction of the subject's gait in relation to one or both legs of the subject 80. In some embodiments, the predicted gait 94 may be a time series data corresponding to the gait of the subject 80 in a predetermined duration. In some embodiments, the predicted gait 94 may be a time series data corresponding to one or more steps of the subject 80. In some embodiments, the two-stage machine learning model 200 may be trained based on EEG signals from the subject. In some embodiments, the two-stage machine learning model 200 may include a first machine learning model and a second machine learning model, each trained at different stages based on EEG signals acquired from the subject 80 at different time points. In various embodiments, at least one machine learning model of the two-stage machine learning model 200 may be used for generating a predicted gait of the subject 80. In various embodiments, at least one machine learning model of the two-stage machine learning model 200 may act as a teaching or guiding model for the other machine learning model of the two-stage machine learning model 200.

In some embodiments, the system 100 may further include a gait acquisition module 130. The gait acquisition module 130 may be configured to obtain actual gait data or an acquired gait 94 of the subject 80. In some embodiments, the gait acquisition module 130 may include goniometers 132 for measuring respective hip angles, knee angles, and ankle joint angles of the subject 80. In other embodiments, the gait acquisition module 130 may include other sensors such as a camera, 3D camera, sonar/depth sensors, LiDAR sensors, inertia measurement units (IMUs), accelerometers, strain gauges, linear/rotational displacement sensors, etc.

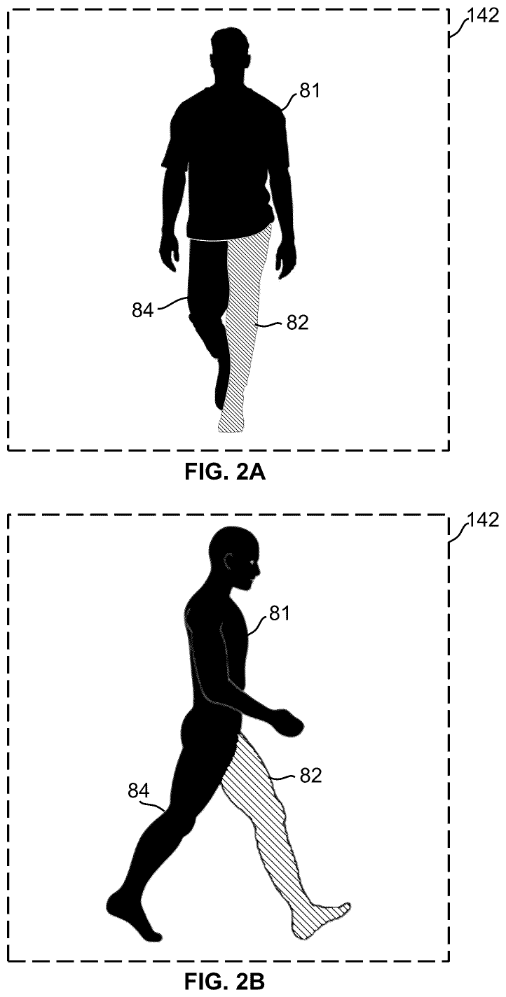

In some embodiments, the system 100 may further include a visual module 140 to provide a visual representation 81 of the subject 80 via a display 142. Referring to FIG. 2A and 2B, in some examples, the visual representation 81 on the display 142 may be an avatar of the subject 80 in gait. For example, the avatar may be displayed in a front view (FIG. 2A) or in a side view (FIG. 2B) or displayed collectively. The visual module 140 may receive the predicted gait 94 from the gait prediction module 110 as well as the acquired gait 96 from the gait acquisition module 130. In some examples, the visual module 140 may provide a visual representation 81 or avatar which includes a visualization of one leg 82 (for example, left leg) of the subject 80 corresponding to the predicted gait 94; and a visualization of another leg 84 (for example, right leg) of the subject 80 corresponding to an acquired gait 96 or actual gait. Therefore, the visual module 140 may be configured to generate an avatar in gait motion based on the predicted gait 94 and the acquired gait 96. The visual feedback aids in providing assistance to the subject 80 for making more accurate motor imagery.

In some embodiments, the processor may be further configured to implement a feature fusion module 150. The feature fusion module 150 may be configured to align the predicted gait 94 and the acquired gait 96 of the subject 80 to obtain gait data of the subject 80. In some embodiments, the gait data may include the predicted gaits 94 stacked in alignment with the acquired gaits 96 (or the actual gait) in the time dimension. In other words, the predicted gaits 94 and the acquired gaits 96 may be synchronized in time. In some embodiments, the gait data may be used for training the feature fusion module 150. The feature fusion module 150 may be further configured to extract and fuse features from the gait data using a deep convolutional neural network, and to determine a gait recovery assessment 98 based on the features extracted. In some embodiments, the feature fusion module 150 may extract selected or specific gait phase(s) of a gait cycle of the subject 80 for gait recovery assessment. In some embodiments, multiple discrete gait phases or gait portions of a gait cycle may be identified and extracted by the feature fusion module 150 for gait recovery assessment. In some embodiments, the dimension of each kernel's output may put a limit on the number of phases each gait cycle is divided into. Therefore, the feature fusion module 150 may be configured to extract the gait phases(s) for gait recovery assessment based on the kernel's output. For example, the feature fusion module 150 may determine the length/duration of each selected gait phase based on the kernel's output. In some embodiments, the convolutional neural network may be trained from gait data acquired from multiple subjects instead of the gait data solely from the subject 80. Therefore, the feature fusion module 150 may be trained with existing data from all subjects before the current subject 80 begins the gait session.

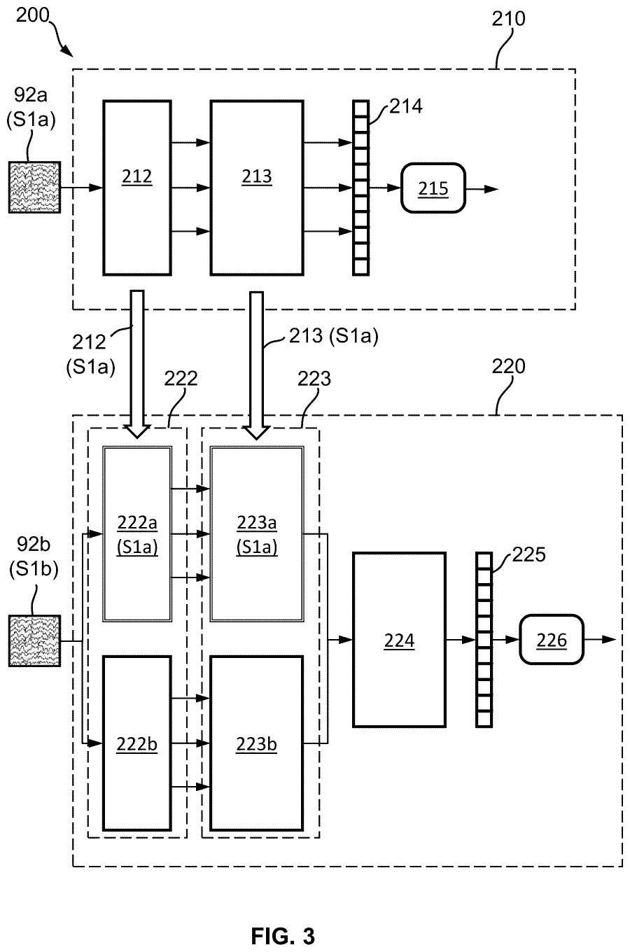

Referring to FIG. 3, in various embodiments, the two-stage machine learning model 200 of the gait prediction module 110 may include a first stage model 210 and a second stage model 220. The first stage model 210 and the second stage model 220 may be trained based on different gait data obtained solely from the subject 80 at different time points. In other words, the training data 92a for the first stage model 210 and the training data 92b for the second stage model 210 are acquired from the subject 80 at different time points. As an example, the training data 92a and 92b may be data acquired from the subject 80 during different gait training sessions or gait assessment sessions. In other examples, the training data 92a/92b acquired from the subject 80 may be data acquired from different portions of the same gait training session or the same gait assessment session.

In some embodiments, the first stage model 210 may include multiple first stage blocks 212/213/214/215. The second stage model 220 may include multiple second stage blocks 222a/222b/223a/223b/224/225/226. Similarly, the first stage blocks 212/213/214/215 and the second stage blocks 222a/222b/223a/223b/224/225/226 may be trained based on different gait data obtained solely from the subject 80 at different time points.

In some embodiments, selected ones of the second stage blocks, such as 222a/223a may be feature extractor blocks. For example, the feature extractor block 222a may be a Temporal Convolution Block (TCB) which is used to learn frequency filters, and the feature extractor block 223a may be a Spatial Convolution Block (SCB) which learns spatial filters using a depth-wise convolution layer. In some examples, the blocks 222a/223a collectively function as a feature extractor for extracting features from the EEG signals.

In some embodiments, each of the feature extractor blocks 222a/223a may correspond to selected ones of the first stage blocks 212/213. In other words, the feature extractor blocks 222a/223a and the respective first stage blocks 212/213 may share similarities with each other. In some embodiments, each of the feature extractor blocks 222a/223a may be identical to the respective first stage blocks 212/213. Therefore, the selected first stage blocks 212/213 may also be feature extractor blocks. For example, feature extractor block 222a may be identical to the first stage block 212, and feature extractor block 223a may be identical to the first stage block 213. Therefore, the feature extractor block 222a may be a copy of or replicated from the respective first stage block 212. It may be understood that in some examples, both the first stage block 212 and feature extractor block 222a are Temporal Convolution Blocks (TCB). In other examples, both the first stage block 213 and feature extractor block 222a are Spatial Convolution Blocks (SCB).

In some embodiments, the feature extractor block 222a may be similar but not identical to the first stage block 212, such as having similar block architecture but slightly differing weights, bias or parameters. For example, the weights of the feature extractor blocks 222a/223a and the respective first stage blocks 212/213 may be in a linear correlation but not identical. In yet other examples, the feature extractor blocks 222a/223a may include weights, bias or parameters identical to that of the selected first stage blocks 212/213, but do not have identical block architectures as the first stage blocks 212/213.

In some embodiments, the two-stage machine learning model 200 may be an attention-based model or include an attention-based functional block. In some embodiments, the second stage model 220 may further include a self-attention block (SAB). The SAB is provided in consideration of data/EEG signals from different time points often contain redundant or less relevant information. Therefore, the SAB is configured to consider the information of all the time points and assign weights to different time points based on importance, which helps to mitigate the deterioration of training performance caused by excessively large tap-size in the training process.

In some embodiments, the second stage model 210 may include pairs of parallel blocks 222/223. For example, the parallel block 222 includes one of the feature extractor blocks 222a in parallel with a respective variable block 222b. Similarly, the parallel block 223 includes another of the feature extractor block 223a in parallel with another respective variable block 223b. The variable block 222b/223b may include similar functionality and/or architecture as the feature extractor blocks 222a/223a. During the operation or training of the second stage model 210, the EEG signals 92b are input into the feature extractor block 222a and the respective variable block 222b concurrently. In some embodiments, the feature extractor blocks 222a/223a and the respective variable blocks 222b/223b of the respective parallel blocks 222/223 are of an identical block architecture. For example, both of the feature extractor block 222a and variable block 222b may be Temporal Convolution Blocks. In another example, both of the feature extractor block 223a and variable block 223b may be Spatial Convolution Blocks. In some embodiments, during training of the second stage model, the respective weights, bias or parameters of each feature extractor blocks 222a/223a are fixed and the respective weights, bias or parameters of each of the variable blocks 222b/223b are updateable. In some embodiments, the second stage model 220 may include a concatenation block 214 for receiving respective outputs from the pair of parallel blocks 223.

Still referring to FIG. 3, in various embodiments, training of the two-stage machine learning model 200 in a single gait session may be described as follows. The first stage model 210, which includes the first stage blocks 212/213/214/215, may be trained with EEG signals 92a collected from the subject 80 in an initial stage S1a of the gait session. For example, the EEG signals 92a may be data collected in the first 10 minutes of the gait session. In some examples, the initial stage S1a corresponds to a calibration phase of the gait session. Upon completion of training, the first stage model 210 may be verified with other EEG signals obtained from the subject 80 in the same gait session or from other sessions. Thereafter, selected ones of the first stage blocks 212 (S1a)/213 (S1a ) may be copied or replicated in the second stage model 220 as corresponding second stage blocks 222a (S1a)/223a (S1a). The selected first stage blocks 212 (S1a)/213 (S1a) may include feature extraction functionality. The references S1a in the paratheses represents the EEG signals 92a the respective blocks 212/213 were trained with. For ease of referencing, the second stage blocks 222a (S1a)/223a (S1a) may be referred to as feature extractor blocks 222a/223a.

Thereafter, training proceeds to the second stage model 220, which includes the second stage blocks 222a/222b/223a/223b/224/225/226. The second stage model 220 may be trained with EEG signals 92b collected from the subject 80 in a later stage S2a of the same gait session. At this instant, the feature extractor blocks 222a/223a of the second stage model 220 corresponds to the selected first stage blocks 212/213 trained with EEG signals 92a. In some embodiments, the feature extractor blocks 222a/223a or parameters of the feature extractor blocks 222a/223a may be fixed during training of the second stage model 230. It may be appreciated that by fixing the feature extractor blocks, the training effect on the second stage model 220 from deteriorating due to the training data's time variation may be avoided. The parameters of the feature extractor blocks 222a/223a may include weights, bias or parameters of the respective blocks. During training of the second stage model 220, only the other second stage blocks 222b/223b/224/225/226 are updateable. In some embodiments, for each gait training session, the first stage model 210 and the second stage model 220 are trained sequentially. In other words, the EEG signals for training the first stage model 210 and the EEG signals for training the second stage model 220 do not overlap eachother during each of the gait training session. For example, the first stage blocks 212/213/214/215 may be trained using EEG signals acquired at the beginning of a gait training session, this followed by the second stage blocks 222a/222b/223a/223b/224/225/226 trained using EEG signals acquired in a subsequent period of the same gait training session.

Upon completion of training, the second stage model 220 may be verified with other EEG signals obtained from the subject 80 in the same gait session or from other sessions. The two-stage machine learning model 200 may be ready for predicting gait based on EEG signals for gait training purposes. As an example, the EEG signals acquired from the subject 80 may be input into the second stage model 240 for extracting gait features from the EEG signals acquired from the subject 80 during a gait training session, and for determining a predicted gait based on the gait features. In some embodiments, the two-stage machine learning model 200 may be a subject-specific model which may be iteratively trained, updated and improved with each passing gait training session.

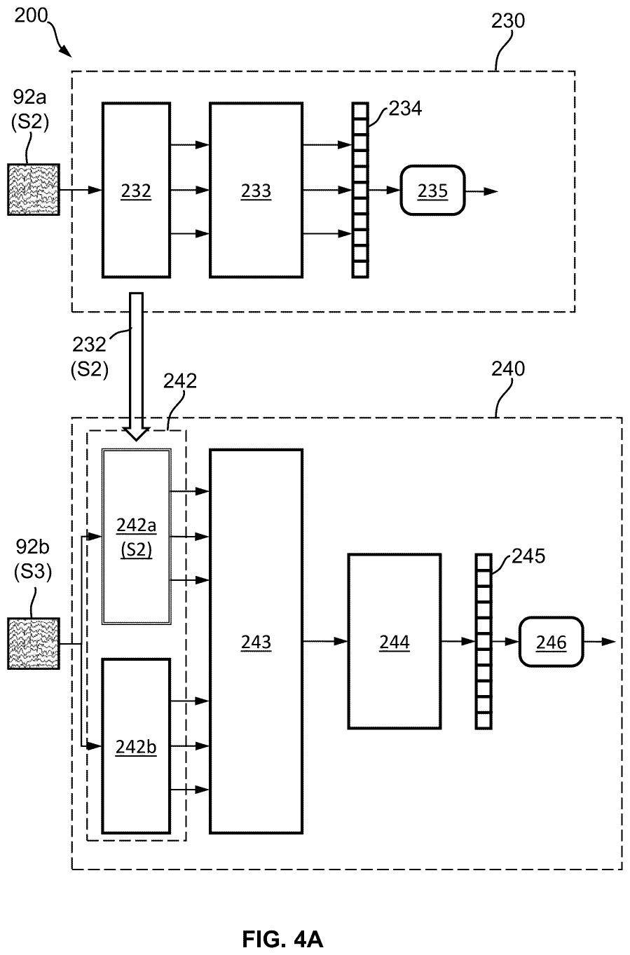

Referring to FIGS. 4A and 4B, in various embodiments, the two-stage machine learning model 200 may include a first stage model 230 and a second stage model 240. The first stage model 230 and the second stage model 240 may be trained based on different gait data obtained solely from the subject 80 at different time points. In some examples, the training data 92a/92b/92c acquired from the subject 80 may be data acquired from different gait training sessions. In some embodiments, the first stage model 230 may include multiple first stage blocks 232/233/234/235. The second stage model 240 may include multiple second stage blocks 242a/242b/243/244/245/246.

In some embodiments, one of the second stage blocks 242a may be a feature extractor block corresponding to first stage block 232. For example, the feature extractor block 242a and respective first stage block 232 may be configured for extracting features from the EEG signals. In some embodiments, the feature extractor block 242a may be identical to the respective first stage blocks 232. In other examples, the feature extractor block 242a may be similar to the respective first stage block 232, such as having identical block architecture but slightly differing weights, bias or parameters, or may include identical weights, bias or parameters identical and slightly differing block architecture.

Still referring to FIGS. 4A and 4B, training of the two-stage machine learning model 200 based on multiple gait sessions may be described as follows. The first stage model 230 may first be trained with EEG signals 92a collected from the subject 80 in one gait session S2. Thereafter, the selected first stage blocks 232 (S2) may be transferred or replicated in the second stage model 240 as corresponding second stage blocks 242a (S2). The selected first stage blocks 232(S2) may include feature extraction functionality. The reference S2 in the paratheses represents the EEG signals 92a the respective blocks 232 are trained with. For ease of referencing, the second stage blocks 242a (S2) may be referred to as feature extractor blocks 242a.

Thereafter, training proceeds to the second stage model 240, which includes the second stage blocks 242a/242b/243/244/245/246. The second stage model 220 may be trained with EEG signals 92b collected from the subject 80 in another gait session S3. At this instant, the feature extractor block 242a of the second stage model 240 may correspond to or may be identical to the first stage block 232 trained with the EEG signals 92a from gait session S2. The feature extractor blocks 242a or parameters of the feature extractor blocks 242a may be fixed during training of the second stage model 230. The parameters of the feature extractor blocks 242a may include weights, bias or parameters of the respective blocks. During training of the second stage model 230, only the other second stage blocks 242b/243/244/245/246 are updateable while the feature extractor blocks 242a are fixed. Therefore, the feature extractor block 242a of the second stage model 230 for a present gait training session S3 may correspond to the respective selected first stage block 232 of the first stage model 230 from a previous gait training session S2.

Further referring to FIG. 4B, in some embodiments, the EEG signals 92b from the gait session S3 may also be used to further train the first stage model 230. Hence, the selected first stage block(s) 232 may be trained over multiple gait training sessions. This enables the selected first stage block 232 with feature extractor functionality to be continuously updated or trained based on EEG signals 92b from subsequent gait sessions. Thereafter, training proceeds to the second stage model 240, in which the second stage model 220 may be trained with EEG signals 92c collected from the subject 80 in a further gait session S4. At this instant, the feature extractor block 242a of the second stage model 240 corresponds to the first stage block 232 trained with the EEG signals 92b from gait session S3. The training process of the two-stage machine learning model 200 may be iteratively trained and improved based on multiple gait sessions.

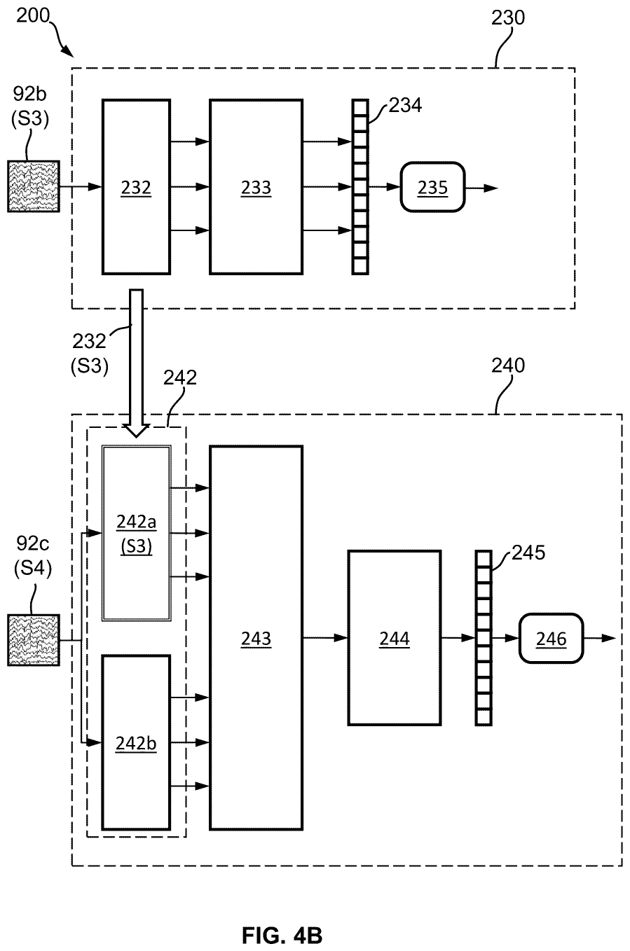

FIG. 5 illustrates a method of gait training 700 according to various embodiments. The method 700 may include in stage 710, using a two-stage machine learning model, extracting gait features from EEG signals acquired from a subject; and in stage 720, using the two-stage machine learning model, determining a predicted gait based on the gait features. In some embodiments, the two-stage machine learning model includes multiple first stage blocks and multiple second stage blocks, the first stage blocks and the second stage blocks are trained based on different gait data obtained solely from the subject at different time points. In some embodiments, at least one of the second stage blocks is a feature extractor block, each of the feature extractor block corresponding to respective ones of the first stage blocks. In avoidance of doubt, using the two-stage machine learning model may include using at least a part of the two-stage machine learning model for extracting gait features from the EEG signals acquired from the subject and/or for determining the predicted gait based on the gait features. For example, only the second stage blocks may be used to generate or determine the gait features and/or predicted gait of the subject based on the EEG signals.

In some embodiments, the method 700 further includes in stage 730, aligning the predicted gait and an acquired gait of the subject to obtain a gait data; and in stage 740, determining a gait recovery assessment based on features extracted from the gait data using a convolutional neural network. In some embodiments, the method may further include providing a visual representation of the subject, the visual representation comprising a visualization of one leg of the subject corresponding to the predicted gait; and a visualization of another leg of the subject corresponding to an acquired gait. In some embodiment, the method 700 may further include training the first stage blocks over multiple gait training sessions.

In some embodiments, the method 700 further includes training the first stage blocks and the second stage blocks based on different gait data obtained solely from the subject at different time points. In some embodiments, the method 700 further includes replicating bias, parameters or weights of selected first stage blocks to the respective second stage blocks.

### EXAMPLES

Various exemplary embodiments of the gait training system are disclosed as follows. The EEG acquisition module 120 may be utilized to acquire multi-channel active EEG and EOG data of the subject 80, and to collect and label the EEG and EOG data in accordance with the extended 10-20 international system. In some examples, the data may be collected at 100 Hz sampling frequency. Baseline correction may be performed to remove linear drift from the EEG/EOG signals or data. The eye artefacts may be removed with vertical Electrooculography (VEOG) and horizontal Electrooculography (HEOG) using Covariance. The EEG/EOG data may be passed through a bandpass filter, such as a filter allowing through a frequency of 0.1 Hz to 49.9 Hz, to remove low and high-frequency noises.

The processed EEG data may include raw EEG signals with C channels with t time points, and joint angle data with dimension d_{J} with t time points. Tap size T refers to the number of time points in history utilized to train the model. The EEG signal may be separated into shorter time segments using a sliding window of size T with hop size 1 along the time dimension. Each segment may be regarded as one input sample for the regressor. Thus, the raw EEG signal may produce (t−T+1) samples. For each sample, the data may be a collection of raw EEG signals with C channels and T time points, denoted by X^{EEG}∈R^{C×T}. The corresponding label d_{J} joint angles of the time window's last time point are expressed as y∈R^{dJ}. The samples may be used to train the feature extraction model.

In some examples, the gait acquisition module 130 may include six goniometers for recording the hip, knee, and ankle joint angles on both sides of the legs at 100Hz sampling frequency. Before each session of data collection begins, the goniometers may be calibrated as follows:
i) zeroing: the subject is standing straight, where the hip, knee, and ankle goniometer measurements are reset to 0°.
ii) getting the maxima: the subject's hips and knees are measured using a goniometer while the subject is seated in a chair (90°), and the subject's feet are rested on an inclined platform) (43°). The readings from these measurements are later utilized as the maximum angles. Due to the physical limitations of human ankles, a platform may be used for ankle joints.
iii) linear mapping: the range of motion for each joint are linearly interpolated between the zero and maximum angle end-points using a mapping technique, such as linear mapping. For example, if the maximum angle of a joint is determined in Step (ii) as “x” degrees, a measurement from this sensor would be linearly mapped to

$$\\ {y}_{map} = \frac{y}{x} \times {x}_{0}$$

where x₀ is 90 degrees for the hip and knee joints, and 43 degrees for the ankle joints. The re-referenced gait data array is split into short time segments, using a sliding window of length, T and hop size 1, as X_{Gait}∈R^{dJ×T}.

In some examples, the visual module 140 may include a visual representation of an avatar on the screen in front of the subject 80, with the avatar controllable by the subject. In the initial stage or calibration process, the avatar's two legs may be controlled with the actual gait of the subject. In the actual gait training process, the left leg of the avatar may still be controlled by the actual gait, while the right leg may be controlled by the predicted gait of the subject's EEG signal with the model.

In some examples, a feature fusion module 150 may also be provided. The predicted gaits may be stacked to align the actual gait in the time dimension. Using a convolutional neural network, the features of the subjects' actual gait and predicted gait may be extracted. The features extracted from the gait data may be used by the feature fusion module to assess the gait rehabilitation of the subject.

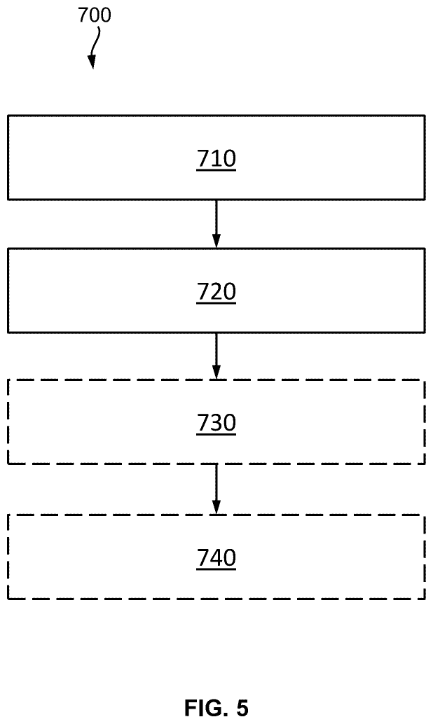

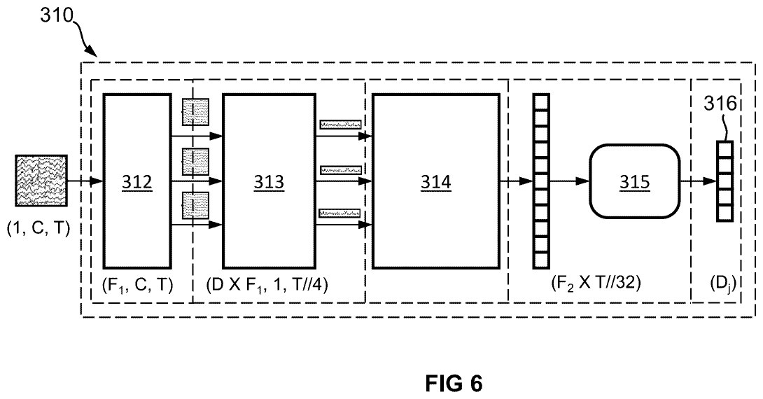

FIGS. 6 and 7 illustrate the two-stage machine learning model or a Multi-model Attention Network (MATN) model according to various examples. The MATN model may extract non-linear features from EEG signals acquired from a subject, and provides an output of the model as a prediction of the subject's gait or predicted gait. The MATN model or two-stage machine learning model may include a first stage model 310 or a teacher model, and a second stage model 320 or a student model.

The first stage model 310 or teacher model may include four machine learning blocks or four functional components. The first block may be a Temporal Convolution Block (TCB) 312 functioning as frequency filters. The second block may be a Spatial Convolution Block (SCB) 313 functioning as a spatial filters using a depthwise convolution layer. The third block may be a separable convolution block 314 which is a combination of a depthwise convolution and a pointwise convolution. The depthwise convolution may function as or learn a temporal summary of each feature map, while the point wise convolution mixes the feature maps. In comparison to conventional convolution, separable convolution may reduce the number of trainable parameters. The last block may be a Fully Connected (FC) layer 315 for joining all the features, and creating an output 316 with the same dimensions as the kinetics labels.

The second stage model 320 or the student model may include five machine learning blocks or five functional components. Similar to the teacher model, the first block may be a Temporal Convolution Block (TCB) 322 which included parallelly connected TCB 322a and 322b. Each of the TCB 322a/322b may receive respective inputs from the same EEG signal. TCB block 322a may a copy or replicate of the TCB 312 of the teacher model. Similarly, the second block may be a Spatial Convolution Block (SCB) 323 which includes parallelly connected SCB 323a and 323b. SCB 323a may be a copy or replicate of the SCB 313. In this example, an output of TCB 322a may be connected to an input of SCB 323a, and similarly, an output of TCB 322b may be connected to an input of SCB 323b. A concatenation block 324 may be provided to receive outputs from the SCB 323a and 323b. Further, a Self-Attention Block (SAB) 325 may be disposed between the SCB 323 and a separable convolution block 326, with a temporary self-attention mechanism being employed in the SAB 325. Since different time points often contain redundant or less relevant information, the SAB 325 may be configured to consider the information of all the time points and assign weights to different time points based on importance. This may assist in mitigating the deterioration of training performance caused by excessively large tap-size in the training process. The separable convolution block 326 may be a combination of a depthwise convolution and a pointwise convolution. Lastly, an output from the separable convolution block 326 may be provided to a Fully Connected (FC) layer 327 which provides an output 328.

The MATN is set up to train two models at different stages with data from different sessions or time points. In this way, the teacher model may be effectively trained to extract the features of the data with large time variations from the test data. The trained feature extractor may be applied to the student model to avoid the training effect being deteriorated due to the training data's time variation. In the first training stage, only the teacher model is trained with data from one or more time points. By the end of the first training stage, the TCB and SCB of the teacher model may have acquired a valid feature extraction strategy, which would be useful in the second training stage. In the second training stage, the parameters of the teacher model's TCB and SCB are fixed, and only the student model's parameters are trainable. The input data or EEG signals are sent to the two TCBs simultaneously and to the corresponding SCBs. The output of the two SCBs would be concatenated and transposed, and passed to the student model's SAB. The EEG signal 324a after concatenation 324 means that multi-channel data of each time point is regarded as an integral feature after the transpose. The SAB's output is transposed and passed to the separable convolution block. In the end, an FC layer maps the output to the kinematic dimension to get the final predicted label. The output of MATN is the gait prediction as y∈R^{dJ}. A predict gait or model output is predicted at every time point, starting from the ‘T’th time point.

Still referring to FIGS. 6 and 7, in some specific examples, the Temporal Convolution Block (TCB) may include a convolution module with F1 filters of size (1, 64) and a Batch Normalization layer, outputting F1 feature maps containing the EEG signal at various band-pass frequencies. Following from the TCB, the Spatial Convolution Block (SCB) may learn spatial filters for each temporal filter. For each feature map, a depth parameter D may determine the number of spatial filters to learn (D=1 as shown in FIGS. 6 and 7 for example purposes). In some examples, each SCB may include a depthwise convolution module with D×F1 kernels of size (C, 1), a Batch Normalization layer, an Exponential Linear Unit (ELU) activation layer, an energy layer, and a dropout layer, all of which are organized in this order. The energy layer may compute the average energy of the time points in a time window. There may be no overlap between time windows. Since the average of the supplied data is close to zero, the energy layer may reduce the data dimension while maintaining the information of the supplied data. The energy layer's time window size may be represented by L. The output may be calculated as:

$$\\ \begin{matrix}{X}_{{EL}_{i , j , k}} = \frac{1}{L} {\sum }_{l = 0}^{L - 1} {X}_{{SCB}_{i , j , k \times L + l}}^{{}^{} 2} & ( 1 )\end{matrix}$$

where L is set to be 4 in this example. X_{SCB} is the output of SCB and X_{EL} is the output of the energy layer.

In the teacher model, the output of SCB may be provided to the separable convolution block, which includes two convolution layers, a Batch Normalization layer, an ELU activation layer, a Max Pooling layer of size 8, and a dropout layer in this order. The first depthwise convolution layer may be F2 kernels of size (1,16), followed by a pointwise convolution layer with F2 kernels of size (1, 1). Following the separable convolution block, features may be passed via the FC layer.

In the student model, the concatenation of outputs from both SCBs may be transposed and passed to the SAB in the student model. The SAB learns three weight matrixes W_{q}, Wₖ, and Wᵥ during training to get query Q, key K, and value V . The output of the block may be calculated as:

$$\\ \begin{matrix}{Self}_{Attention ( X )} = Softmax {}^{} ( \frac{{QK}^{{}^{} T}}{\sqrt{{d}_{k}}} ) {}^{} V = Softmax {}^{} ( \frac{{W}_{q} {X ( {W}_{k} X )}^{{}^{} T}}{\sqrt{{d}_{k}}} ) {W}_{v} & ( 2 )\end{matrix}$$

where dₖ in MATN is set as 2×F2 to align X and Q, K, V.

Similar to the teacher model, the separable convolution block in the student model includes a depthwise convolution layer, a pointwise convolution layer, a Batch Normalization layer, an ELU activation layer, a Max Pooling layer of size 8, and a dropout layer, in order. Due to dimension change of input data, the first depthwise convolution layer includes (2×F2) kernels of size (1,16), and the pointwise convolution layer has (2×F2) kernels of size (1, 1).

### EXPERIMENTS

In the experiments, the MATN and two variants of MATN were compared with baseline models such as Linear Regression, LSTM, CNNLSTM, TCN, EEGNet. The first variant of the MATN was a Multi-Model Network (MMN), which included the two-stage model architecture but did include a SAB. The second variant was a Self-Attention Model (SAM), which corresponds to the student model of MATN but did not include a teacher model or two-stage model architecture.

The mobile brain-body imaging (MoBI) dataset (a lower limb motor imagery dataset) was used as the dataset for the experiments. Radiofrequency (RF) wireless interface (MOVE system, Brain Products GmbH, Germany) was used to collect EEG data/signals from the subjects, and six goniometers were employed to record bilateral joint angles on the legs (hip, knee, and ankle) at 100 Hz. Eight healthy individuals participated in the experiments. Each subject was put through three trials on two different days. The first two trials were conducted on the first day, while the third trial happened on the other day. The subject were asked to walk on the treadmill for 20 minutes in each trial. In the first 15 minutes, the avatar on the screen in front of the subject would provide real-time feedback based on the actual joint angles and the decoder would be trained. In the next 5 minutes, the avatar's right leg would be controlled by the EEG signal of the subject, providing real-time feedback on the screen. The subjects were asked to stay still for 2 minutes before and after the 20-minute walking.

Curry7 commercial software was used to process the raw data. First, a baseline correction was performed to remove linear drift. Thereafter, the eye artifacts with VEOG and HEOG were removed using Covariance. Next, we passed the data through a bandpass filter from 0.1 Hz to 49.9 Hz. For problematic blocks were replaced with a signal that varies uniformly and monotonically with time. Finally, bad channels were repaired using the interpolate bads( ) method embedded in the open toolkit MNE (Python).

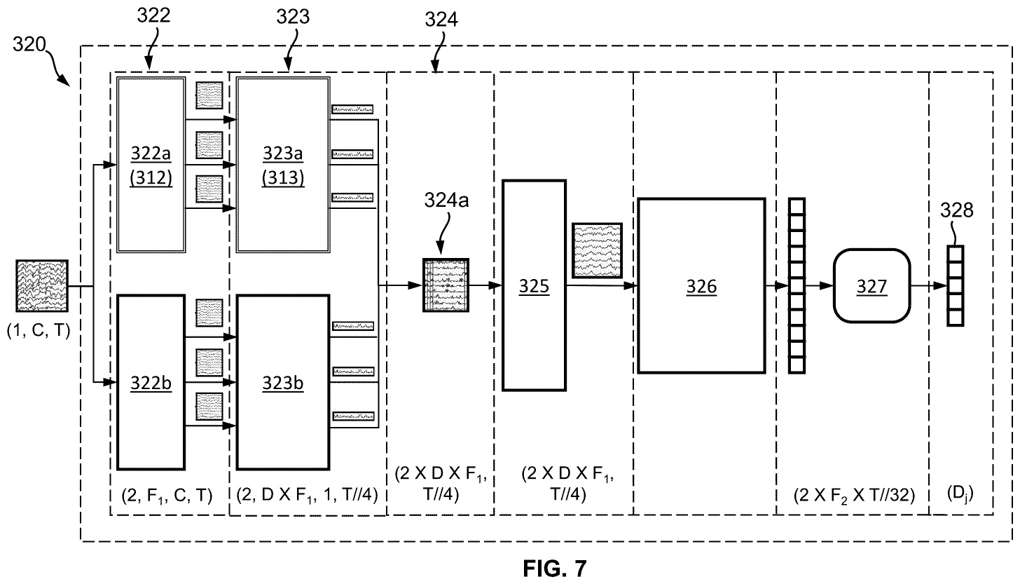

FIG. 8 illustrates the partition of EEG signal/data for the experiments. For the MATN, the visible data before the test phase included the 20-minutes walking data from trials 1 and 2, as well as the first 15-minutes walking data from trial 3. The test set included the last 5-minutes of walking data from trial 3. The first 13.5-minutes of walking data from both trials 1 and 2 made up the training set/data for the MATN's teacher model. The remaining data (6.5-minutes) of trials 1 and 2 were used as the validation set/data. For MATN's student model, the training set included the first 13.5-minutes of walking data from trial 3, and the validation set included the subsequent 1.5-minutes of walking data from trial 3. For the other baseline models, the training set was composed of the first 13.5 minutes of walking data from trials 1, 2, and 3. The validation set includes the subsequent 6.5-minutes of walking data from trials 1 and 2, as well as the subsequent 1.5-minutes of walking data from trial 3.

The deep learning methods were implemented using the PyTorch library. Two experiments were performed for each baseline deep learning model with different hyperparameters. The hyperparameters and number of trainable parameters of each model is indicated in Table I. The MATN model was trained using Adam optimizer at default settings. Mean-square-error loss was adopted for gradient update. The batch size was set as 100. The maximum of epoch epochmax was set as 100. The training patience p was set as 30 which indicated stopping training when the Pearson's correlation coefficient (r-value) of the validation set had not increased for 30 epochs.

TABLE I
Models' hyperparameters and numbers of trainable
parameters (tap size = 100)

| | | Number of |
|---|---|---|
| Algorithm | Hyperparameter | Parameters |
| CNNLSTM | S = 5, H = 64, L = 2 | 54876 |
| | S = 4, H = 32, L = 2 | 14910 |
| LSTM | H = 32, L = 2 | 20678 |
| | H = 64, L = 2 | 7270 |
| TCN | N = 4, H = 64, K = 10 | 52326 |
| | N = 2, H = 16, K = 10 | 18486 |
| EEGNet | F₁ = 8, D = 2, F₂ = 16 | 2358 |
| | F₁ = 16, D = 4, F₂ = 64 | 11430 |
| SAM | F₁ = 8, D = 2, F₂ = 16 | 3446 |
| | F₁ = 16, D = 2, F₂ = 32 | 9446 |
| MMN | F₁ = 8, D = 2, F₂ = 16 | 6060 |
| | F₁ = 16, D = 2, F₂ = 32 | 14668 |
| MATN | F₁ = 8, D = 2, F₂ = 16 | 10796 |

A comparison of performance between the models was performed, to show the respective performance of the models when trained with data from only trial 3 in comparison to trials 1, 2 and 3. MATN and MMN both included teacher and student models which require data from different sessions. The outcome is shown in Table II.

Referring to Table II, the difference column was calculated as difference between r-values of trials1+2+3 and r-values of tria13. It may be observed that for baseline models, the difference was under 0, this indicated a deterioration in the performance of the baseline deep learning models as more data (EEG data) was added to the training set. The reason for this may be due to the fact that the data from trials 1 and 2 differ from trial 3 in terms of distribution as the trial data were obtained on different dates. Even though the training set of all three trials covered the entire training set of a single trial, the varied distributions might have worsened the performance. This highlighted the advantage of the MATN model which mitigated the performance deterioration caused by diverse data distributions. When the difference was greater than 0, this indicated an improvement in the performance of the MATN and the MMN model when more data of other trials were added to the training set. In comparison to the baseline models, MATN and MMN models made more effective use of data from different sessions due to the multi-model structure.

TABLE II
The average r-value of different algorithms trained with
different amounts of training data (tap size = 100).

Training Data

| | | trial 1 + | | Number of |
|---|---|---|---|---|
| Algorithm | trial 3 | 2 + 3 | Difference | Parameters |
| Linear | 0.620 | 0.579 | −0.141 | |
| CNNLSTM | 0.577 | 0.512 | −0.065 | 54876 |
| | 0.63 | 0.565 | −0.065 | 14910 |
| LSTM | 0.596 | 0.540 | −0.056 | 20678 |
| | 0.627 | 0.536 | −0.091 | 7270 |
| TCN | 0.674 | 0.615 | −0.059 | 52326 |
| | 0.605 | 0.507 | −0.098 | 18486 |
| EEGNet | 0.686 | 0.609 | −0.077 | 2358 |
| | 0.691 | 0.599 | −0.092 | 11430 |
| SAM | 0.718 | 0.612 | −0.096 | 3446 |
| | 0.727 | 0.592 | −0.135 | 9062 |
| MMN | | 0.716 | +0.025 | 6060 |
| | | 0.696 | +0.005 | 14668 |
| MATN | | 0.734 | +0.017 | 10796 |

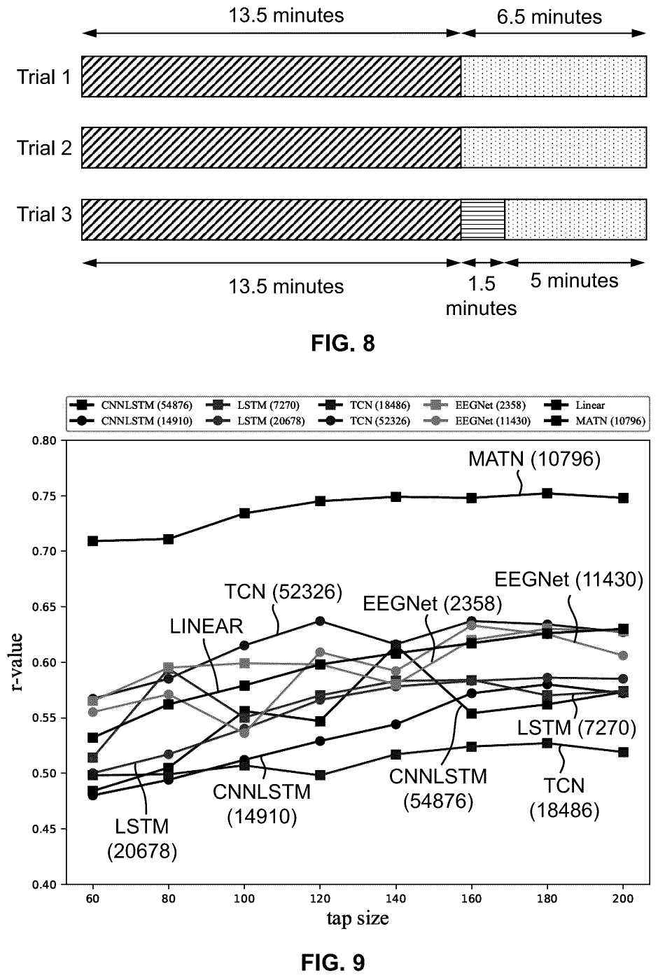

FIG. 9 shows a comparison of r-value of MATN and the baseline models. In the legend of FIG. 9, the figures in the parantheses represent the number of trainable parameters. As shown in FIG. 9, the performance of the baseline deep learning models did not grow monotonically or converge as the tap size was increased. It may be due to the fact that when the time window contained excessive time points with a low correlation to the motion of the last time point, the models continued to treat all the time points equally meaningful. Thus, resulted in a large amount of noise which detrimentally affected the training effect of the model. To address such problem, previous approaches often include conducting more experiments to discover a local maximum as an ideal tap size. In the proposed system/method of the present disclosure, a self-attention mechanism was employed in the time domain, motivating the model to automatically determine the importance of every time point, this mitigating the performance deterioration. For the experiments carried out using the MoBI dataset in a subject-specific manner, the MATN model achieved the highest Pearson's correlation coefficient (more than 18% higher) in comparison to the baseline models, such as Linear Regression, LSTM, CNNLSTM, TCN, and EEGNet.

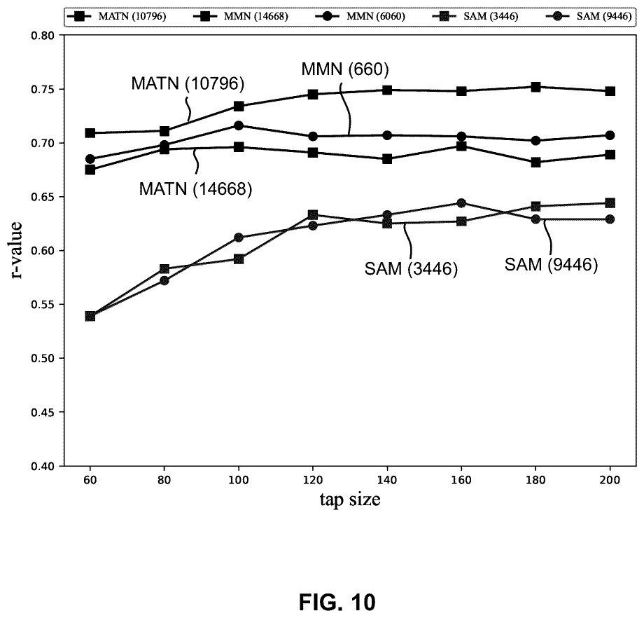

FIG. 10 shows that the regression effect of the proposed model is not only better than the baseline models but also converges gradually with the increase of tap size. Further, FIG. 10 shows the results of an ablation study based on the MATN model and the two variant models (MMN and SAM). In the ablation study, MMN used the same separation strategy of training and validation set as in MATN. In the experiment employing SAM, the separation method was identical with the baseline models trained with trial 1+2+3. As self-attention mechanism was applied in SAM, it may be seen that the r-value converged with the tap size increasing. However, since SAM does not take into consideration a different distribution of data, the respective r-values were significantly lower than that of MATN. As for MMN which adopted the teacher-student model to train the network with data of different sessions at different training stages, the regression effect rose markedly. Nevertheless, without the self-attention mechanism, MMN was not able to handle the temporal dynamics of EEG signals pertaining to individual subjects, leading to a relatively lower robustness for large tap size in comparison to MATN.

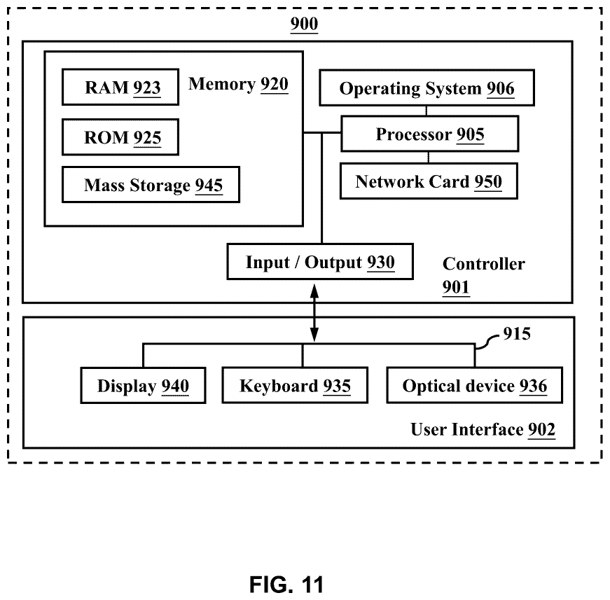

In accordance with embodiments of the present disclosure, a block diagram representative of components of processing system 900 that may be provided within PID controller module 104 to carry out the functions of the PID controller module, or any other modules of the system is illustrated in FIG. 11. One skilled in the art will recognize that the exact configuration of each processing system provided within these modules may be different and the exact configuration of processing system 900 may vary and the arrangement illustrated in FIG. 9 is provided by way of example only.

In embodiments of the invention, processing system 900 may comprise controller 901 and user interface 902. User interface 902 is arranged to enable manual interactions between a user and the computing module as required and for this purpose includes the input/output components required for the user to enter instructions to provide updates to each of these modules. A person skilled in the art will recognize that components of user interface 902 may vary from embodiment to embodiment but will typically include one or more of display 940, keyboard 935 and optical device 936.

Controller 901 is in data communication with user interface 902 via bus 915 and includes memory 920, processor 905 mounted on a circuit board that processes instructions and data for performing the method of this embodiment, an operating system 906, an input/output (I/O) interface 930 for communicating with user interface 902 and a communications interface, in this embodiment in the form of a network card 950. Network card 950 may, for example, be utilized to send data from these modules via a wired or wireless network to other processing devices or to receive data via the wired or wireless network. Wireless networks that may be utilized by network card 950 include, but are not limited to, Wireless-Fidelity (Wi-Fi), Bluetooth, Near Field Communication (NFC), cellular networks, satellite networks, telecommunication networks, Wide Area Networks (WAN) and etc.

Memory 920 and operating system 906 are in data communication with CPU 905 via bus 910. The memory components include both volatile and non-volatile memory and more than one of each type of memory, including Random Access Memory (RAM) 923, Read Only Memory (ROM) 925 and a mass storage device 945, the last comprising one or more solid-state drives (SSDs). One skilled in the art will recognize that the memory components described above comprise non-transitory computer-readable media and shall be taken to comprise all computer-readable media except for a transitory, propagating signal. Typically, the instructions are stored as program code in the memory components but can also be hardwired. Memory 920 may include a kernel and/or programming modules such as a software application that may be stored in either volatile or non-volatile memory.

Herein the term “processor” is used to refer generically to any device or component that can process such instructions and may include: a microprocessor, microcontroller, programmable logic device or other computational device. That is, processor 905 may be provided by any suitable logic circuitry for receiving inputs, processing them in accordance with instructions stored in memory and generating outputs (for example to the memory components or on display 940). In this embodiment, processor 905 may be a single core or multi-core processor with memory addressable space. In one example, processor 905 may be multi-core, comprising—for example—an 8 core CPU. In another example, it could be a cluster of CPU cores operating in parallel to accelerate computations.

All examples described herein, whether of apparatus, methods, materials, or products, are presented for the purpose of illustration and to aid understanding, and are not intended to be limiting or exhaustive. Modifications may be made by one of ordinary skill in the art without departing from the scope of the invention as claimed.

1. A system for gait training, comprising:
memory storing instructions; and
a processor coupled to the memory and configured to process the stored instructions to implement:
a gait prediction module configured to:
using a two-stage machine learning model, extract gait features from EEG signals acquired from a subject; and determine a predicted gait based on the gait features,
wherein the two-stage machine learning model includes multiple first stage blocks and multiple second stage blocks, the first stage blocks and the second stage blocks being trained based on different gait data obtained solely from the subject at different time points,
wherein at least one of the second stage blocks is a feature extractor block, each of the feature extractor block corresponding to respective ones of the first stage blocks.

2. The system as recited in claim 1, wherein the second stage blocks include a self-attention block (SAB).

3. The system as recited in claim 1, wherein the processor is further configured to implement:
a feature fusion module configured to:
align the predicted gait and an acquired gait of the subject to obtain a gait data; and
determine a gait recovery assessment based on features extracted from the gait data using a convolutional neural network.

4. The system as recited in claim 3, wherein the convolutional neural network is trained from gait data acquired from multiple subjects.

5. The system as recited in claim 1, wherein each of the feature extractor block is identical to the respective first stage block.

6. The system as recited in claim 1, wherein the first stage blocks and the second stage blocks are trained sequentially in each gait training session.

7. The system as recited in claim 1, wherein each of the feature extractor block of a present gait training session corresponds to respective ones of the first stage blocks from a previous gait training session.

8. The system as recited in claim 1, wherein the first stage blocks are trained over multiple gait training sessions.

9. The system as recited in claim 1, wherein during training of the second stage blocks, parameters of each of the feature extractor block are fixed.

10. The system as recited in claim 1, wherein the second stage blocks comprise at least one pair of parallel blocks, each of the at least one pair of parallel blocks comprises one of the feature extractor block in parallel with a respective variable block, wherein the EEG signals are input into each of the feature extractor block and the respective variable block concurrently.

11. The system as recited in claim 10, wherein the feature extractor block and the respective variable block of each pair of parallel blocks are of an identical block architecture.

12. The system as recited in claim 10, wherein during training of the second stage blocks, parameters of each of the feature extractor block are fixed and parameters of each of the variable block are updateable.

13. The system as recited in claim 10, wherein the second stage blocks include a concatenation block for receiving respective outputs from one of the at least one pair of parallel blocks.

14. The system as recited in claim 1, wherein the feature extractor block is configured as at least one of: a Temporal Convolution Block (TCB) and a Spatial Convolution Block (SCB).

15. The system as recited in claim 1, wherein the processor is further configured to implement:
a visual module configured to:
provide a visual representation of the subject, the visual representation comprising
a visualization of one leg of the subject corresponding to the predicted gait; and
a visualization of another leg of the subject corresponding to an acquired gait.

16. The system as recited in claim 1, further comprising: a gait acquisition module for obtaining an acquired gait of the subject, wherein the gait acquisition module includes goniometers for measuring hip angles, knee angles, and ankle joint angles of the subject.

17. A method of gait training, comprising:
using a two-stage machine learning model,
extracting gait features from EEG signals acquired from a subject; and
determining a predicted gait based on the gait features,
wherein the two-stage machine learning model includes multiple first stage blocks and multiple second stage blocks, the first stage blocks and the second stage blocks being trained based on different gait data obtained solely from the subject at different time points,
wherein at least one of the second stage blocks is a feature extractor block, each of the feature extractor block corresponding to respective ones of the first stage blocks.

18. The method as recited in claim 17, further comprising:
aligning the predicted gait and an acquired gait of the subject to obtain a gait data; and
determining a gait recovery assessment based on features extracted from the gait data using a convolutional neural network.

19. The method as recited in claim 17, further comprising:
providing a visual representation of the subject, the visual representation comprising a visualization of one leg of the subject corresponding to the predicted gait; and a visualization of another leg of the subject corresponding to an acquired gait.

20. The method as recited in claim 17, further comprising training the first stage blocks over multiple gait training sessions.
